SEQUENCE LISTING (9 sequences)

SEQ ID NO: 1 - protein, 8 aa, synthetic construct
  REGION 1..8: note = PEPTIDE
  MOD_RES 1: note = X is pyroglutamic acid
XYDGKGVG

SEQ ID NO: 2 - protein, 9 aa, synthetic construct
  REGION 1..9: note = PEPTIDE
  PEPTIDE 6..7: note = The sixth Cys and the seventh Gly are both bound to the fifth Lys.
QYDGKCGVG

SEQ ID NO: 3 - protein, 8 aa, synthetic construct
  REGION 1..8: note = PEPTIDE
  MOD_RES 1..7: note = The first X is pyroglutamic acid. The fourth and seventh X can be any naturally occurring amino acid.
XYDXKGXG

SEQ ID NO: 4 - protein, 8 aa, synthetic construct
  REGION 1..8: note = PEPTIDE
  MOD_RES 1: note = X is pyroglutamic acid.
XFDGKGVG

SEQ ID NO: 5 - protein, 8 aa, synthetic construct
  REGION 1..8: note = PEPTIDE
  MOD_RES 1: note = X is pyroglutamic acid.
XYEGKGVG

SEQ ID NO: 6 - protein, 8 aa, synthetic construct
  REGION 1..8: note = PEPTIDE
  MOD_RES 1: note = X is pyroglutamic acid.
XYDSKGVG

SEQ ID NO: 7 - protein, 8 aa, synthetic construct
  REGION 1..8: note = PEPTIDE
  MOD_RES 1: note = X is pyroglutamic acid.
XYDGKSVG

SEQ ID NO: 8 - protein, 8 aa, synthetic construct
  REGION 1..8: note = PEPTIDE
  MOD_RES 1: note = X is pyroglutamic acid.
XYDGKGLG

SEQ ID NO: 9 - protein, 8 aa, synthetic construct
  REGION 1..8: note = PEPTIDE
  MOD_RES 1: note = X is pyroglutamic acid.
XYDGKGVS

(19) United States
(12) Patent Application Publication
(10) Pub. No.: US 2025/0172570 A1
(43) Pub. Date: May 29, 2025

(54) IMMUNOASSAY METHOD FOR CROSS-LINKED N-TELOPEPTIDE OF TYPE I COLLAGEN, IMMUNOASSAY KIT, AND ANTIBODY OR ANTIBODY FRAGMENT THEREOF

(71) Applicant: SEKISUI MEDICAL CO., LTD., Tokyo (JP)

(72) Inventors: Tomo SHIMIZU, Tokyo (JP); Tomohide ASAI, Tokyo (JP); Satoko SEO, Tokyo (JP); Rikako OGASAWARA, Tokyo (JP)

(73) Assignee: SEKISUI MEDICAL CO., LTD., Tokyo (JP)

(21) Appl. No.: 18/688,920

(22) PCT Filed: Oct. 18, 2022
(86) PCT No.: PCT/JP2022/038704

§ 371 Date: Mar. 4, 2024

(30) Foreign Application Priority Data
Oct. 20, 2021 (JP) 2021-171927

(51) Int. Cl. G01N 33/68; C07K 16/18; G01N 33/543

(52) U.S. Cl. CPC G01N 33/6887; C07K 16/18; G01N 33/54326; G01N 33/54386; C07K 2317/33; G01N 2333/78

(57) ABSTRACT

An immunoassay method for cross-linked N-telopeptide of type I collagen, comprising a step of contacting a biological sample with an antibody or antibody fragment thereof that binds to a peptide fragment having an amino acid sequence represented by JYDGKGVG (SEQ ID NO: 1). This method is easy in operation and enable NTx measurement with higher accuracy.

### SEQUENCE LISTING

A sequence listing in electronic (XML file) format is filed with this application and incorporated herein by reference. The name of the XML file is “Sequence_Listing_0367”; the file was created on Nov. 3, 2024; the size of the file is 15,394 bytes.

### TECHNICAL FIELD

The present invention relates to an immunoassay method and immunoassay kit for cross-linked N-telopeptide of type I collagen. The present invention also relates to an antibody or an antibody fragment thereof.

### BACKGROUND ART

Cross-linked N-telopeptide of type I collagen (hereinafter also referred to as NTx) is a bone-derived degradation product of type I collagen. NTx is produced when type I collagen is digested by Cat K during the process of bone resorption. After being produced, NTx is excreted into the blood and/or urine.

The level of NTx increases as the bone resorption advances. Therefore, NTx serves as an index that directly reflects bone resorption. This allows NTx to be used as a marker for diagnosing osteoporosis or determining therapeutic efficacy.

Patent Literature 1 describes that the rate of bone resorption is measured by measuring NTx in urine. Patent Literature 2 describes monoclonal antibody 1H11 that binds to NTx. Further, Patent Literature 2 also describes an epitope recognized by the monoclonal antibody 1H11.

A kit for measuring NTx by ELISA method using a monoclonal antibody is also commercially available (Non-Patent Literature 1). However, it has been desired to develop a measuring method for NTx, which is easier in operation and enables measurement with higher accuracy.

### CITATION LIST

### Patent Literature

Patent Literature 1: Japanese Unexamined Patent Application Publication (Translation of PCT Application) No. H3-500818
Patent Literature 2: Japanese Unexamined Patent Application Publication (Translation of PCT Application) No. H11-505804

### Non Patent Literature

Non-Patent Literature 1: Package insert of in vitro diagnostic drug “Osteomark (registered trademark) Kit for Cross-linked N-telopeptides of Type I Collagen” (Abbott Diagnostics Medical Co., Ltd.)
Non-Patent Document 2: J. Bone Miner. Res 1992, 7 (11), 1251-1258 A specific immunoassay for monitoring human bone resorption: quantitation of type I collagen cross-linked N-telopeptides in urine

### SUMMARY OF INVENTION

### Technical Problem

An object of the present invention is to provide a measuring method for NTx, which is easier in operation and enables NTx measurement with higher accuracy.

### Solution to Problem

The present inventors have found that the amount of uric acid present in the measurement system during NTx measurement affects the measured value of NTx. Biological samples such as urine and serum used as samples for NTx measurement contain uric acid. Further, a urine sample containing a high NTx concentration needs to be diluted with urine with a known NTx concentration. The resulting diluted urine also contains uric acid. The concentration of uric acid in biological samples or urine for dilution varies. Therefore, the measured value of NTx is affected by the amount of uric acid contained in such biological samples or urine for dilution, which may hinder accurate measurement of NTx.

The present inventors have made extensive studies and confirmed that using an antibody that binds to a peptide fragment having an amino acid sequence JYDGKGVG (SEQ ID NO: 1) (J is pyroglutamic acid) enables an immunoassay where the measured value of NTx is less likely to be affected even when uric acid is present during NTx measurement.

Patent Literature 2 describes that even when monoclonal antibody 1H11 and a peptide fragment having an amino acid sequence represented by JYDGKGVG (SEQ ID NO: 1) were assayed, no commercially meaningful antibody binding value was obtained. Further, Non-Patent Literature 2 discusses that the epitope for the monoclonal antibody 1H11 is not a linear peptide simply synthesized from the individual sequences of α1 and α2 N-telopeptides. Furthermore, Non-Patent Literature 2 also discusses that epitopes are assumed to exist in the three-dimensional structure of a specific cross-linked peptide sequence. This is presumably because the monoclonal antibody 1H11 uses NTx as an immunogen.

That is, the present inventors have found that the above-mentioned problem can be solved by using an antibody having a reactivity different from that of the monoclonal antibody 1H11.

The present invention is as follows.
<1> An immunoassay method for cross-linked N-telopeptide of type I collagen, comprising a step of contacting a biological sample with an antibody or antibody fragment thereof that binds to a peptide fragment having an amino acid sequence represented by

(SEQ ID NO: 1)
JYDGKGVG.

<2> The immunoassay method according to <1>, wherein the antibody or the antibody fragment thereof binds to a peptide fragment having an amino acid sequence represented by QYDGK (C) GVG (SEQ ID NO: 2).
<3> The immunoassay method according to <1> or <2>, wherein the antibody or the antibody fragment thereof is a monoclonal antibody or an antibody fragment thereof.
<4> The immunoassay method according to any one of <1> to <3>, wherein the biological sample is urine, blood, plasma, or serum.
<5> The immunoassay method according to any one of <1> to <4>, wherein a concentration of uric acid contained in a measurement system in the step of contacting is 0.001 to 0.1% by mass.
<6> The immunoassay method according to any one of <1> to <5>, wherein the step of contacting is a step of bringing the biological sample into contact with the antibody or the antibody fragment thereof in the presence of a peptide fragment for detection which has an amino acid sequence represented by JYDX₁KGX₂G (SEQ ID NO: 3), wherein the X₁ and the X₂ are predetermined amino acids,
   wherein the peptide fragment for detection is bound to a solid phase or a labeling substance, and
   the antibody or the antibody fragment thereof is allowed to bind to the peptide fragment for detection.

<7> The immunoassay method according to <6>, wherein the X₁ is glycine or serine, and the X₂ is valine or leucine.
<8> The immunoassay method according to any one of <1> to <7>, which further comprises a step of measuring a signal generated by the labeling substance,
wherein the peptide fragment for detection is bound to a solid phase, and
the antibody or the antibody fragment thereof is indirectly or directly bound to the labeling substance.
<9> The immunoassay method according to <8>, wherein the solid phase is magnetic particles and the labeling substance is a ruthenium complex.
<10> An immunoassay kit for cross-linked N-telopeptide of type I collagen in a biological sample, comprising an antibody or an antibody fragment thereof that binds to a peptide fragment having an amino acid sequence represented by JYDGKGVG (SEQ ID NO: 1).
<11> The immunoassay kit according to <10>, wherein the antibody or the antibody fragment thereof binds to a peptide fragment having an amino acid sequence represented by QYDGK(C)GVG (SEQ ID NO: 2).
<12> The immunoassay kit according to <10> or <11>, wherein the antibody or the antibody fragment thereof is a monoclonal antibody or an antibody fragment thereof.
<13> The immunoassay kit according to any one of <10> to <12>, wherein the biological sample is urine, blood, plasma, or serum.
<14> The immunoassay kit according to any one of <10> to <13>, further comprising a peptide fragment for detection containing an amino acid sequence represented by

(SEQ ID NO: 3)
JYDX₁KGX₂G,

wherein the X₁ and the X₂ are predetermined amino acids, and
the antibody or the antibody fragment thereof binds to the peptide fragment for detection.
<15> The immunoassay kit according to <14>, wherein the X₁ is glycine or serine, and the X₂ is valine or leucine.
<16> The immunoassay kit according to any one of <10> to <15>, wherein the peptide fragment for detection is bound to a solid phase, and the antibody or the antibody fragment thereof is indirectly or directly bound to the labeling substance.
<17> The immunoassay kit according to <16>, wherein the solid phase is magnetic particles and the labeling substance is a ruthenium complex.
<18> An antibody or antibody fragment thereof that binds to a peptide fragment having an amino acid sequence represented by JYDGKGVG (SEQ ID NO: 1).
<19> The antibody or antibody fragment thereof according to <18>, which binds to a peptide fragment having an amino acid sequence represented by QYDGK(C)GVG (SEQ ID NO: 2).
<20> The antibody or antibody fragment thereof according to <18> or <19>, which is a monoclonal antibody.
<21> A solid phase for use in the immunoassay method of any one of <1> to <9>, which has a peptide fragment for detection bonded thereto, wherein the peptide fragment contains an amino acid sequence represented by JYDX₁KGX₂G (SEQ ID NO: 3) or a partially modified sequence thereof, wherein the X₁ and the X₂ are predetermined amino acids.
<22> The solid phase according to <21>, which is magnetic particles.
<23> The solid phase according to <21> or <22>, wherein the X₁ is glycine or serine, and the X₂ is valine or leucine.

### Advantageous Effects of Invention

The present invention can provide a NTx measuring method and a NTx measuring kit, which are easier in operation and enable NTx measurement with higher accuracy. The present invention can also provide an antibody or an antibody fragment thereof that can be used in the measuring method and the measuring kit.

### BRIEF DESCRIPTION OF DRAWINGS

FIG. 1 is a diagram showing the chemical structure of NTx.

FIG. 2 is a diagram showing an embodiment of the immunoassay method of the present invention.

FIG. 3 is a diagram showing the results of antigen-immobilized ELISA (solid phase: N×7 peptide or N×2 peptide, antibody: S88230R).

FIG. 4 is a diagram showing the results of antigen-immobilized ELISA (solid phase: N×7 peptide or N×2 peptide, antibody: 1H11).

### DESCRIPTION OF EMBODIMENTS

Hereinbelow, the descriptions are given separately for various aspects of the invention, but the descriptions, definitions of terms, and embodiments described for a particular aspect are also applicable to the other aspects.

### 1. Immunoassay Method for Cross-linked N-telopeptide of Type I Collagen

### Cross-linked N-telopeptide of Type I Collagen (NTx)

Cross-linked N-telopeptide of type I collagen is a bone-derived degradation product of type I collagen. NTx is produced when type I collagen is digested by Cat K during the process of bone resorption. After being produced, NTx is excreted into the blood and/or urine.

The level of NTx increases as the bone resorption advances. Therefore, NTx serves as an index that directly reflects bone resorption. This allows NTx to be used as a marker for diagnosing osteoporosis or determining therapeutic efficacy.

In addition to diagnosing osteoporosis or determining therapeutic efficacy, NTx is also measured for the following purposes.
Determination of the suitability for surgery for primary hyperparathyroidism
Evaluation of therapeutic effects after hyperparathyroidism surgery
Indicators of bone metastasis of malignant tumors and indicators of the degree of progression of bone metastatic lesions

In this context, the immunoassay method of the present invention is not limited to those implemented for the above purposes.

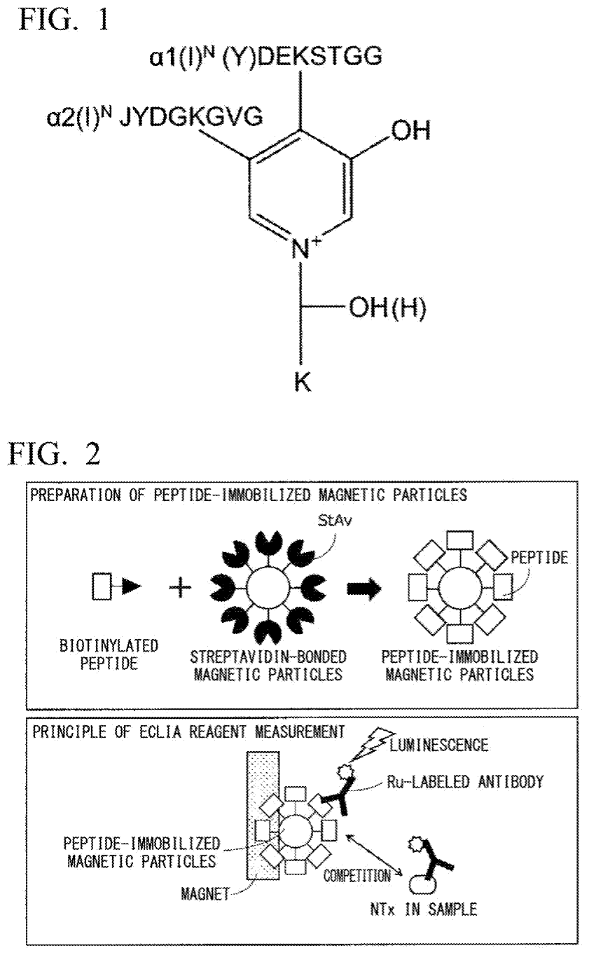

NTx has a structure shown in FIG. 1. In NTx, an α1 chain and an α2 chain are bonded to a pyridinium crosslinked structure. The α2 chain has an amino acid sequence represented by JYDGKGVG (SEQ ID NO: 1).

### Biological Sample

Examples of the “biological sample” in the present specification include solid tissues and body fluids derived from living bodies.

The biological sample is preferably a body fluid, examples which include blood, serum, plasma, urine, tears, ear discharge, prostatic fluid, and respiratory secretions. The biological sample is preferably urine, blood, plasma, or serum, more preferably urine or serum, and even more preferably urine.

Examples of subjects from which the biological sample is to be collected include humans or animals (e.g., monkeys, dogs, and cats), of which humans are preferable. The biological sample may be a biological sample as it is taken from a subject, or may be a sample obtained by subjecting a collected biological sample to treatments such as dilution and concentration that are usually performed. The person who collects and prepares a biological sample used in the present invention may or may not be identical to the person who performs the immunoassay method of the present invention. Further, the biological sample used in the immunoassay method of the present invention may be one collected or prepared during implementation of the immunoassay method of the present invention, or one previously collected or prepared and stored.

The biological sample may be one collected from a subject suffering from a metabolic disease that causes increased bone resorption, such as osteoporosis, primary hyperparathyroidism, or a malignant tumor with suspected bone metastases (particularly breast, lung, or prostate cancer).

### Antibody

In the immunoassay method of the present invention, monoclonal antibodies or polyclonal antibodies can be used as long as the effects of the present invention can be obtained. In the immunoassay method of the present invention, it is preferable to use a monoclonal antibody. In the immunoassay method of the present invention, an antibody fragment possessing the function of antibody can also be used as long as the effects of the present invention can be achieved. Further, in the immunoassay method of the present invention, the antibody may be of any isotype (IgM, IgD, IgG, IgA, or IgE) as long as the effects of the present invention are obtained, but IgG is preferred.

### Monoclonal Antibody

In the context of the present specification, the term “monoclonal antibody” refers to an antibody or antibody molecule that is obtained from clones derived from a single antibody-producing cell. In the immunoassay method of the present invention, an antibody fragment possessing the function of monoclonal antibody can also be used as long as the effects of the present invention can be achieved. Examples of antibody fragments possessing the function of monoclonal antibody include a functional fragment including the Fab portion of a monoclonal antibody obtained by enzymatic digestion of the monoclonal antibody, a functional fragment including the Fab portion of a monoclonal antibody produced by genetic recombination, a functional fragment including scFv produced by phage display method, and the like.

### Labeling Substance

It is preferable that a labeling substance is bound to the antibody used in the present invention. By measuring the intensity of the signal generated by the labeling substance, the amount of NTx in the biological sample can be measured. The labeling substance may be directly bound to the antibody used in the present invention or indirectly bound to the antibody via a secondary antibody. Hereinbelow, an antibody used in the present invention, to which a labeling substance is bound, may be referred to as a labeled antibody. Examples of labeling substances for preparing the labeled antibody include metal complexes, enzymes, insoluble particles, fluorescent substances, chemiluminescent substances, electrochemiluminescent substances (such as ruthenium complexes), biotin, avidin, radioactive isotopes, colloidal gold particles, and colored latex. The method used for binding the labeling substance to the antibody may be physical adsorption, glutaraldehyde method, maleimide method, pyridyl disulfide method, or periodic acid method, which are available to those skilled in the art.

In the immunoassay method of the present invention, as the labeling substance, it is preferable to use an electrochemiluminescent substance, and it is more preferable to use a ruthenium complex. When an enzyme such as horseradish peroxidase (HRP) or alkaline phosphatase (ALP) is used as a labeling substance, enzymatic activity can be measured using a specific substrate for the enzyme. When the enzyme is HRP, for example, O-phenylenediamine (OPD) or 3,3′,5,5′-tetramethylbenzidine (TMB) can be used, and in the case of ALP, p-nitrophenyl phosphate and the like can be used.

In the context of the present specification, the process of physically or chemically supporting an antigen or antibody on a solid phase or the state of an antigen or antibody being supported on a solid phase is sometimes referred to as “immobilization” or “solid-phase immobilization”. The “assay”, “detection” or “measurement” of NTx also encompasses determination of the presence or absence of NTx, and quantification of NTx.

### Peptide Fragment Having an Amino Acid Sequence Represented by JYDGKGVG (SEQ ID NO: 1)

The antibody or antibody fragment thereof used in the immunoassay method of the present invention binds to a peptide fragment having an amino acid sequence represented by JYDGKGVG (SEQ ID NO: 1). Hereinbelow, the antibody or antibody fragment thereof that binds to the peptide fragment having an amino acid sequence represented by JYDGKGVG (SEQ ID NO: 1) may be referred to as the antibody of the present invention. The antibody of the present invention preferably also binds to a peptide fragment having an amino acid sequence represented by QYDGK(C)GVG (SEQ ID NO: 2), as well as the peptide fragment having an amino acid sequence represented by JYDGKGVG (SEQ ID NO: 1). The (C) in “QYDGK(C)GVG” is bonded to the side chain of K. That is, the “(C)” and the first “G” in “GVG” are both bonded to K.

The antibody of the present invention may be an isolated antibody or an antibody fragment thereof, preferably an isolated monoclonal antibody or an antibody fragment thereof.

The antibody of the present invention preferably specifically binds to a peptide fragment containing an amino acid sequence represented by JYDGKGVG (SEQ ID NO: 1). The expression “specifically binds to” means that the antibody or antibody fragment thereof does not substantially bind to peptide fragments other than those having a specific amino acid sequence.

More preferably, the antibody of the present invention does not substantially bind to either the α1 chain portion or the pyridinium bridge structure in NTx.

The antibody of the present invention preferably binds to a peptide fragment having an amino acid sequence represented by QYDGK(C)GVG (SEQ ID NO: 2) and/or a peptide fragment having an amino acid sequence represented by an JYDX₁KGX₂G (SEQ ID NO: 3). In SEQ ID NO: 3, X₁ and X₂ are predetermined amino acids, and X₁ is preferably glycine or serine, while X₂ is preferably valine or leucine. More preferably, X₁ is glycine and X₂ is valine.

In order to determine whether the antibody of the present invention “does not substantially bind” to a peptide fragment having a certain amino acid sequence, for example, it is possible to perform a measurement based on the SPR method, using Biacore (registered trademark) T100 or T200, while having the antibody of the present invention being immobilized. Whether the monoclonal antibody “does not substantially bind” can also be determined by other methods or means well known to those skilled in the art than the above SPR method.

### Peptide Fragment for Detection

When a competitive method is employed in the immunoassay method of the present invention, it is preferable that the biological sample and the antibody of the present invention are brought into contact with each other in the presence of a peptide fragment for detection containing an amino acid sequence represented by JYDX₁KGX₂G (SEQ ID NO: 3). Since the antibody of the present invention binds to both the α2 chain in NTx and the peptide fragment for detection, there is a competition between binding of the antibody of the present invention to the α2 chain in NTx, and binding of the antibody of the present invention to the peptide fragment for detection (see FIG. 2). The length of the peptide fragment for detection is, for example, 50 amino acids or less, 30 amino acids or less, 20 amino acids or less, or 10 amino acids or less. The peptide fragment for detection is preferably a peptide fragment having an amino acid sequence represented by JYDX₁KGX₂G (SEQ ID NO: 3). The peptide fragment for detection may be bound to either a solid phase or a labeling substance, but is preferably bound to a solid phase.

The order of adding the biological sample, the antibody of the present invention, and the peptide fragment for detection to the measurement system is not limited as long as the effects of the present invention can be obtained.

When the immunoassay method of the present invention is performed by a competitive method in the presence of a peptide fragment for detection, the antibody of the present invention binds to the peptide fragment for detection. The peptide fragment for detection may include an amino acid sequence represented by JYDX₁KGX ₂G (SEQ ID NO: 3). In SEQ ID NO: 3, X₁ and X₂ are predetermined amino acids, and X₁ is preferably glycine or serine, while X₂ is preferably valine or leucine. More preferably, X₁ is glycine and X₂ is valine.

The peptide fragment for detection may be prepared by synthesis, or NTx or the α2 chain in NTx may be extracted and used. The peptide fragment for detection may be an α2 chain as a genetically recombinant protein, or a genetically recombinant protein containing JYDX₁KGX₂G (SEQ ID NO: 3).

For example, a solid phase bound to a peptide fragment for detection can be produced by allowing the peptide fragment for detection to physically adsorb or chemically bind to the solid phase (possibly via an appropriate spacer). It is preferable to use streptavidin and biotin for binding the solid phase and the peptide fragment for detection. Specifically, streptavidin is bound to the solid phase, and then the peptide fragment for detection and biotin are bound thereto. Next, the solid phase and the peptide fragment for detection can be bound by binding the streptavidin and biotin. The binding between the peptide fragment for detection and biotin is preferably carried out via the amino group of lysine contained in the peptide fragment.

The solid phase to be used may be a solid phase composed of a polymer substrate such as polystyrene resin, an inorganic substrate such as glass, a polysaccharide substrate such as cellulose or agarose, or the like. The shape of the solid phase is not particularly limited, and any appropriate shape may be chosen, for example, from a plate shape (e.g., microplate or membrane), a bead or particulate shape (e.g., latex particles, magnetic particles), or a cylindrical shape (e.g., test tube).

In the immunoassay method of the present invention, when a monoclonal antibody is used, it is preferable to use only a single type of monoclonal antibody for immunoassay of NTx, for example, by employing the competition method described below. When using two types of monoclonal antibodies, the two types of monoclonal antibodies preferably recognize different epitopes. To recognize different epitopes in this context means that the amino acid sequences recognized as epitopes by the two monoclonal antibodies do not overlap. When there are multiple epitopes, one of the multiple epitopes only needs to be different from one of the multiple epitopes of the other antibody.

The present specification includes descriptions indicating that an antibody or an antibody fragment thereof “reacts with”, “recognizes” or “binds to” a specific substance or amino acid sequence, which however are used interchangeably. Whether or not an antibody “reacts with” an antigen (compound) can be determined through antigen-immobilized ELISA, competitive ELISA, sandwich ELISA, or the like. Alternatively, a method using the principle of surface plasmon resonance (SPR method) can also be used. The SPR method can be performed using equipment, sensors and reagents commercially available under the name Biacore (registered trademark).

For example, when performing the same operation as the screening method of Preparation Example 1 (antigen-immobilized ELISA) described below, a peptide fragment with its absorbance significantly increased compared to a negative control without addition of a peptide fragment can be evaluated as being bound to the antibody.

### Method for Preparing Monoclonal Antibody

When using a monoclonal antibody in the immunoassay method of the present invention, the monoclonal antibody can be prepared by dissolving a peptide fragment having an amino acid sequence represented by QYDGK(C)GVG (SEQ ID NO: 2) as an antigen (immunogen) in a solvent such as in phosphate buffered saline and administering the resulting solution to a non-human animal for immunization. Immunization may be performed using an emulsion after addition of an appropriate adjuvant to the solution as necessary. Examples of adjuvants include generally used adjuvants, such as water-in-oil emulsions, water-in-oil-in-water emulsions, oil-in-water emulsions, liposomes, and aluminum hydroxide gels, as well as proteins or peptide substances derived from biological components.

The type of animal used for immunization is also not particularly limited, and mammals such as mice, rats, cows, rabbits, goats, sheep, or alpacas are preferred, and mice or rats are more preferred. Animals can be immunized following common techniques, for example, by subcutaneously, intracutaneously, intravenously, or intraperitoneally injecting an antigen solution, preferably a mixture thereof with an adjuvant, into the animals. Since the immune response generally differs depending on the type and strain of the animal to be immunized, it is desirable to appropriately set the immunization schedule according to the animal used. It is preferable to repeat the antigen administration several times after the initial immunization.

For obtaining the monoclonal antibody of the present invention, the following operations may be subsequently performed, which, however, are not essential and do not limit the present invention. Methods for producing monoclonal antibodies per se are well known and widely used in the art, and those skilled in the art can prepare the monoclonal antibody of the present invention by using the antigens as described above (see, for example, Antibodies, A Laboratory Manual (Cold Spring Harbor Laboratory Press, (1988), Chapter 6, etc.).

After the final immunization, antibody-producing spleen cells or lymph node cells are extracted from the immunized animal and fused with a myeloma-derived cell line having high proliferative potential to prepare hybridomas. Cells with high antibody-producing ability (in terms of quality and quantity) are preferably used for cell fusion, and it is more preferable that the myeloma-derived cell line is compatible with the animal from which the antibody-producing cells to be fused are derived. Cell fusion can be performed according to methods known in the art.

After the cloning step, the ability of the produced monoclonal antibody to bind to the peptide fragment having the amino acid sequence represented by QYDGK(C)GVG (SEQ ID NO: 2) can be assayed by methods such as ELISA, RIA, and immunofluorescence. These operations allow determination of whether the selected hybridomas produce monoclonal antibodies with the desired properties.

By mass-culturing the hybridomas selected as described above, monoclonal antibodies having the desired properties can be produced. The method for mass-culturing is not particularly limited, and examples thereof include a method that cultures hybridomas in an appropriate medium to produce monoclonal antibodies in the medium, and a method that injects hybridomas into the peritoneal cavity of a mammal to allow the hybridomas to proliferate, thereby producing monoclonal antibodies in ascites.

As the monoclonal antibody, it is possible to use an antibody fragment of a monoclonal antibody having antigen-antibody reactivity as well as the whole antibody molecule. In addition to those obtained through the process of immunizing animals as described above, it is also possible to use monoclonal antibodies obtained using gene recombination techniques, such as chimeric antibodies, humanized antibodies and human antibodies. Examples of fragments of monoclonal antibodies include F(ab′)₂, Fab′, scFv, and the like. These fragments can be prepared by treating the monoclonal antibody obtained as described above with a protease (e.g., pepsin or papain), or cloning the DNA of the antibody and expressing it in a culture system using E. coli or yeast.

### Uric acid

In the immunoassay method of the present invention, the measurement system may include uric acid. Uric acid is an organic compound represented by a molecular formula C₅H₄N₄O₃ (CAS RN 69-93-2).

The concentration of uric acid during the antibody-antigen reaction is not limited as long as the effects of the present invention can be obtained, but the concentration is, for example, 0.001 to 0.1% by mass, preferably 0.01 to 0.1% by mass.

In the present specification, the amount of uric acid can be measured by an “enzymatic method” using uricase, which is a urate oxidase.

### Immunoassay Method

The “immunoassay method” is a method of measuring the level of a substance contained in a biological sample using the reaction between an antigen and an antibody. The term “level” encompasses the amount, concentration, or determination of presence or absence of a substance.

Examples of the immunoassay method of the present invention include, but not limited to, electrochemiluminescence immunoassay (ECLIA method), enzyme-linked immunosorbent assay (ELISA), latex immunoturbidimetric assay (LTIA method), chemiluminescence immunoassay, immunochromatography, and immunofluorescence. The immunoassay method of the present invention is preferably ELISA or ECLIA.

The immunoassay method of the present invention can be an in vivo or in vitro immunoassay method. Further, a sensitizer may also be used to enhance sensitivity.

The concentration of the antibody of the present invention in the measurement system can be adjusted as appropriate depending on the immunoassay method or the type of biological sample. For example, the concentration may be 0.1 ng/ml to 100 μg/mL.

Hereinbelow, the procedure and principle of measurement are explained, taking as examples competitive ELISA and competitive ECLIA as immunoassay methods. The following descriptions are presented for illustrative purpose only with respect to the measurement procedure and principle for one embodiment of the present invention, and by no means limit the scope of the present invention. In each of the immunoassay methods described below, methods known in the art including those described above can be used without any limitation with respect to specific methods such as the method for immobilizing the antibody on the solid phase, the method for binding the antibody to the labeling substance, and the type of labeling substance.

The immunoassay method of the present invention may be, for example, competitive ELISA, which is a competitive method, including steps (1) to (3) as described below. The order of performing the steps (1) to (3) is not limited.
(1) A biological sample to be analyzed is added to a microplate on which a peptide fragment for detection is immobilized.
(2) An enzyme-labeled antibody of the present invention is added to the microplate.
(3) A substrate for the enzyme is added, and a signal attributable to the enzymatic reaction is measured.

The signal intensity decreases when NTx is present in the biological sample, and competition occurs between the following two reactions: a reaction between NTx in the biological sample and the antibody of the present invention, and a reaction between the peptide fragment for detection immobilized on the solid-phase and the antibody of the present invention. Further, the antibody may be labeled with biotin instead of an enzyme. In this case, streptavidin labeled with an enzyme may be bound to the biotin. Then, a chromogenic signal generated by addition of OPD, etc. as a substrate can be measured.

A secondary antibody can also be used in competitive ELISA. In the present specification, the “secondary antibody” is an antibody that specifically recognizes the antibody of the present invention. When using a secondary antibody, the following procedures (1) to (5) can be adopted.
(1) A biological sample to be analyzed is added to a microplate on which a peptide fragment for detection is immobilized.
(2) An antibody of the present invention is added to the microplate.
(3) Further, an enzyme-labeled secondary antibody is added.
(4) A substrate is added for color development.
(5) The signal attributable to the substrate is measured using a plate reader or the like.

Electrochemiluminescence immunoassay (ECLIA) means a method in which a labeling substance is caused to emit light by application of electric current, and the amount of light emitted is detected to measure the amount of a target substance to be detected. A ruthenium complex can be used as a labeling substance in the electrochemiluminescence immunoassay. Radicals generated on the electrodes excite the ruthenium complex, causing it to emit light. Then, the amount of light emitted from this ruthenium complex can be detected.

The immunoassay method of the present invention may be, for example, competitive ECLIA, which is a competitive method, including steps (1) to (3) as described below. The order of performing the steps (1) and (2) is not limited.
(1) A biological sample to be analyzed is added to a measurement system containing magnetic particles on which a peptide fragment for detection is immobilized.
(2) The antibody of the present invention that is labeled with an electrochemiluminescent substance, preferably a ruthenium complex, is added to the measurement system.
(3) The intensity of luminescence derived from the luminescent label is measured. The signal intensity decreases when NTx is present in the biological sample, and competition occurs between the following two reactions: a reaction between NTx in the biological sample and the antibody of the present invention, and a reaction between the peptide fragment for detection immobilized on the magnetic particles and the antibody of the present invention.

The immunoassay method of the present invention may further include the following steps, if necessary:
step of pretreating a biological sample,
step of immobilizing a peptide fragment for detection on a solid phase,
B/F washing step of washing and removing antibodies that are not bound to the peptide fragment for detection as well as the biological sample,
step of calculating the NTx concentration in the biological sample from the measured luminescence intensity, based on the luminescence intensity in a measurement of a sample containing NTx in a known concentration, and/or
comparison step of comparing the calculated NTx concentration in the biological sample with a first threshold value.

Examples of pretreatments include filtration of the biological sample and dilution of the biological sample with a sample diluent.

The first threshold may be appropriately set in consideration of sensitivity, type of the biological sample, and purpose of the NTx measurement. When the biological sample is urine, the following values can be adopted as the first threshold value depending on the purpose of measurement.
Indication for parathyroidectomy: 200 nM BCE/mM-Cre or higher
Indicator of bone metastasis of malignant tumors (breast cancer, lung cancer, prostate cancer): 100 nM BCE/mM-Cre or higher
Indicator of increased bone resorption: 55 nM BCE/mM-Cre or higher
Indicator of osteoporosis drug treatment (indicator of high risk of fracture): higher than 54.3 nM BCE/mM-Cre
Indicator of osteoporosis drug treatment (indicators of high risk of bone loss): 35.3 nM BCE/mM-Cre or higher

The first threshold may be a numerical range. The first threshold being a numerical range means that the specified range include a specific threshold, and the determination of whether the measured value is larger or smaller than the specific threshold allows determination of the presence or absence of a disease, etc.

When the first threshold is a numerical range, the first threshold may be in a range between 1.0 and 300 nM BCE/mM-Cre, between 5.0 and 250 nM BCE/mM-Cre, or between 7.0 and 220 nM BCE/mM-Cre.

In the immunoassay method of the present invention, when the signal intensity is higher than the first threshold, the method may include a step of determining that a subject is suffering from a metabolic disease that causes increased bone resorption, such as osteoporosis or primary hyperparathyroidism, or that bone metastasis is suspected in a subject suffering from a malignant tumor (particularly breast cancer, lung cancer, or prostate cancer).

In the immunoassay method of the present invention, when the signal intensity is lower than the first threshold, the method may include a step of determining that a subject is not suffering from a metabolic disease that causes increased bone resorption, such as osteoporosis or primary hyperparathyroidism, or that bone metastasis is not suspected in a subject suffering from a malignant tumor (particularly breast cancer, lung cancer, or prostate cancer).

Based on the NTx concentration in a biological sample calculated by the immunoassay method of the present invention, the therapeutic effect of a specific drug on a subject suffering from osteoporosis can be determined. In this instance, the immunoassay method of the present invention may further include the following step(s) in addition to the steps described above:
a step of administering a specific drug to the subject, and/or
a step of comparing the NTx concentration in the biological sample collected from the subject with a second threshold.

In this instance, the second threshold may be appropriately set in consideration of sensitivity of the immunoassay method, type of the biological sample, and purpose of the NTx measurement. The second threshold may be the measured NTx in the subject prior to administration of a specific drug to the subject.

The immunoassay method of the present invention may include a step of determining that a specific drug has a therapeutic efficacy when the signal intensity is lower than the second threshold, or a step of determining that a specific drug has no therapeutic efficacy when the signal intensity is higher than the second threshold.

In the determination of the therapeutic efficacy, the therapeutic efficacy may be monitored by conducting measurement every few days.

Examples of the specific drugs include bisphosphonate preparations, anti-RANKL antibodies (denosumab), and calcium preparations.

### 2. Immunoassay Kit for Cross-linked N-Telopeptide of Type I Collagen in Biological Sample

The immunoassay kit for NTx in a biological sample according to the present invention (hereinafter also referred to simply as the immunoassay kit of the present invention) contains the antibody of the present invention.

The immunoassay kit of the present invention may be an immunoassay kit comprising one type of the antibody of the present invention for use in a competitive method, preferably a competitive ELISA or a competitive ECLIA.

Examples of the immunoassay kit of the present invention include, but not limited to, immunoassay kits for performing immunochromatography, ELISA, electrochemiluminescence immunoassay, latex immunoturbidimetry, chemiluminescence immunoassay, and immunofluorescence.

The immunoassay kit of the present invention can be an immunoassay kit for assaying in vivo or in vitro samples.

The immunoassay kit of the present invention can also include other test reagents such as standard antigen substances and quality control antigen samples, specimen diluents, and/or an instruction manual, etc. A person skilled in the art can appropriately adjust the concentrations of the antibody-containing reagent and the like.

Hereinbelow, the reagents contained in the kit are explained, taking as examples competitive ELISA and competitive ECLIA.

For competitive ELISA, the immunoassay kit of the present invention may include the following (A):
(A) the antibody of the present invention.

The antibody (A) of the present invention is preferably labeled with an enzyme.

For competitive ELISA, the immunoassay kit of the present invention preferably includes the following (B) and/or (C) in addition to the above (A).
(B) A peptide fragment for detection containing an amino acid sequence represented by JYDX₁KGX₂G (SEQ ID NO: 3), wherein X₁ and X₂ are predetermined amino acids, and X₁ is preferably glycine or serine, while X₂ is preferably valine or leucine. More preferably, X₁ is glycine and X₂ is valine.
(C) A solid phase for immobilizing (B), which is preferably an immunoplate.

With respect to the peptide fragment (B) for detection, the kit may include the peptide fragment (B) that has been immobilized on the solid phase (C) in advance. When the peptide fragment (B) for detection and the solid phase (C) are included separately in the kit, the person performing the immunoassay is supposed to immobilize the peptide fragment (B) for detection on the solid phase (C).

Further, the immunoassay kit of the present invention may further include (D) a secondary antibody that specifically binds to the antibody of the present invention.

For competitive ECLIA, the immunoassay kit of the present invention may include the following (A):
(A) A labeling reagent containing the antibody of the present invention labeled with an electrochemiluminescent substance (e.g., ruthenium complex).

For competitive ELISA, the immunoassay kit of the present invention preferably includes the following (B) and (C) in addition to the above (A).
(B) A peptide fragment for detection containing an amino acid sequence represented by JYDX₁KGX₂G (SEQ ID NO: 3), wherein X₁ and X₂ are predetermined amino acids, and X₁ is preferably glycine or serine, while X₂ is preferably valine or leucine. More preferably, X₁ is glycine and X₂ is valine.
(C) A solid phase for immobilizing (B), which is preferably magnetic particles.

With respect to the peptide fragment (B) for detection, the kit may include the peptide fragment (B) that has been immobilized on the solid phase (C) in advance. When the peptide fragment (B) for detection and the solid phase (C) are included separately in the kit, the person performing the assay is supposed to immobilize the peptide fragment (B) for detection on the solid phase (C).

Hereinbelow, the present invention is described in detail with reference to examples, which however should not be construed as limiting the present invention. In the following description, the unit “%” refers to “% by mass”, unless otherwise specified.

### EXAMPLES

### Preparation Example 1 Preparation of Monoclonal Antibody

### Preparation of Immunogen and Antigen for Screening

Imject Maleimide-Activated Ovalbumin (manufactured by Thermo Scientific, CAT No. 77126) was dissolved in 0.2 mL of purified water to prepare 10 mg/ml of Maleimide-Activated Ovalbumin solution. 2 mg of peptide QYDGK(C)GVG ((C) is bonded to the side chain of K, Nx-2 peptide) was dissolved in 0.2 mL of PBS to obtain a 10 mg/ml peptide solution. The prepared Maleimide-Activated Ovalbumin solution and the peptide solution were mixed and stirred at room temperature for 2 hours. The resulting reaction solution was dialyzed against PBS to obtain a peptide (immunogen) to which Ovalbumin was bound via the thiol group of cysteine.

### Immunization Method

20 μL of the immunogen was mixed with Freund's Complete Adjuvant (manufactured by Difco Laboratories) and the resulting was used to immunize a 6-week-old F344/Jc1 rat subcutaneously on its back or footpad. Two weeks later, 20 μL of the immunogen was mixed with Freund's Incomplete Adjuvant (manufactured by Difco Laboratories) and the resulting was used to immunize the rat subcutaneously on its back or footpad. The same procedure was repeated continually every two weeks. In the third and subsequent immunizations, the individuals having shown a sufficient increase in antibody titer were intraperitoneally immunized with the immunogen diluted in PBS. One to three days after the intraperitoneal immunization, spleen cells, iliac lymph node cells, and inguinal lymph node cells were collected and fused with myeloma cells SP2/0 by electrofusion method. The fused cells were cultured in a 96-well plate, and the culture supernatant was collected 7 or 8 days after the fusion, followed by screening by antigen-immobilized ELISA described below. The strains that reacted to the Nx-2 peptide were selected and cloned.

### Screening Method (Antigen-immobilized ELISA)

Imject Maleimide-Activated BSA (manufactured by Thermo Scientific, CAT No. 77126) was dissolved in 0.2 mL of purified water to prepare 10 mg/ml of Maleimide-Activated BSA solution. 2 mg of Nx-2 peptide was dissolved in 0.2 mL of PBS to obtain a 10 mg/ml peptide solution. The prepared Maleimide-Activated BSA solution and the peptide solution were mixed and stirred at room temperature for 2 hours. The resulting reaction solution was dialyzed against PBS to obtain a peptide to which BSA was bound via the thiol group of cysteine (i.e., Nx-2 peptide-bound BSA). 50 μL of Nx-2 peptide-bound BSA dissolved in PBS at a concentration of 0.1 μL/mL was dispensed into each well of a 96-well plate and allowed to stand at room temperature for 2 hours. After washing each well three times with PBST, 100 μL of blocking solution (1% BSA-PBST) was dispensed into each well and the resulting was allowed to stand at room temperature for 1 hour. After removing the blocking solution from each well, 50 μL of the culture supernatant was dispensed into each well and the resulting was allowed to stand at room temperature for 1 hour. After washing each well three times with PBST, 50 μL of HRP-labeled goat anti-rat IgG (Fc) polyclonal antibody (manufactured by Southern Biotech) diluted 10,000 times with a blocking solution was dispensed into each well and the resulting was allowed to stand at room temperature for 1 hour. After washing each well three times with PBST, 50 μL of an OPD coloring solution was dispensed into each well and the resulting was allowed to stand at room temperature for 10 minutes. 50 μL of a stop solution was added to each well to stop the reaction. Absorbance at a wavelength of 492 nm was measured using a plate reader to determine the reactivity between the Nx-2 peptide-bound BSA and the antibody. Three strains of antibodies that react with Nx-2 peptide and NTx were established. The S88230R antibody was selected from the established antibodies, and ascites was prepared using antibody-producing cells, which was then purified using a protein G column to be used in subsequent tests.

### Preparation of 1H11 Antibody

HB-10611 hybridoma (ATCC) was cultured, and antibodies were purified from the culture supernatant using a protein A column and used in the subsequent tests.

### Example 1 Evaluation of the Influence of Uric Acid on the NTx Measurement System

### Preparation of Biotin-Labeled Nx7 Peptide

2 mg of a peptide (Nx7 peptide) having an amino acid sequence JYDGKGVG was dissolved in 1 mL of 100 mM PBS with a pH of 7.5 to prepare an Nx7 peptide solution. 12.7 mg of Ez-Link NHS-PEG12-Biotin (manufactured by Thermo Scientific) was dissolved in 0.041 mL of dehydrated DMF, and the entire amount of the resulting was added to the Nx7 peptide solution. After stirring on ice for 3 hours, the resulting was purified by reverse phase chromatography to remove unreacted biotin reagent, thereby obtaining a biotin-labeled Nx7 peptide solution.

### Nx7 Peptide-Immobilized ELISA

After washing Pierce Streptavidin Coated Plates (manufactured by Thermo Scientific) three times with PBST, 50 μL of 0.1 μg/mL biotin-labeled Nx7 peptide was dispensed into each well and the resulting was allowed to stand at room temperature for 1 hour. After washing each well three times with PBST, 50 μL of a solution containing 0.3 or 0.05 μg/mL S88230R antibody or 1H11 antibody and 0.02, 0.05 or 0.10% uric acid was dispensed into each well, and the resulting was allowed to stand at room temperature for 1 hour. After washing each well three times with PBST, 50 μL of HRP-labeled goat anti-rat IgG (H+L) (manufactured by Southern Biotech) diluted 9,500 times with a blocking solution or HRP-labeled goat anti-rat IgG (H+L) (manufactured by Southern Biotech) diluted 8,500 times with a blocking solution was dispensed into each well and the resulting was allowed to stand at room temperature for 1 hour. After washing each well three times with PBST, 50 μL of an OPD coloring solution was dispensed into each well and the resulting was allowed to stand at room temperature for 10 minutes. 50 μL of a stop solution was added to each well to stop the reaction. Absorbance at a wavelength of 492 nm was measured using a plate reader. The absorbance ratios in measurements of the samples were calculated with the absorbance in measurement of a sample without uric acid being defined as 100%.

### NTx-Immobilized ELISA

The same procedure as in the steps after addition of the biotin-labeled Nx7 peptide in the Nx7 peptide-immobilized ELISA in Example 1 was performed, except that the peptide-immobilized plate of the Nx7 peptide-immobilized ELISA was changed to an antigen binding plate of Osteomark kit for cross-linked N-telopeptides of type I collagen (Abbott Diagnostics Medical Co., Ltd.). According to the package insert of the product, the antigen-binding plate contains NTx in an amount that gives a sensitivity of 20 nmol BCE/L per well.

The results are shown in Table 1. When the Nx7 peptide was used as the solid-phase antigen, no change in absorbance was observed in response to an increase in the concentration of uric acid added for any of the antibodies. On the other hand, when the NTx was used as the solid-phase antigen, a decrease in absorbance was observed in response to an increase in the concentration of uric acid added for all antibodies, indicating that the antigen-antibody reaction was inhibited by uric acid. The degree of inhibition of the antigen-antibody reaction by uric acid was smaller in S88230R than in 1H11.

TABLE 1

| | | Absorbance ratio | | | | | | | |
|---|---|---|---|---|---|---|---|---|---|
| | | Immobilized antibody | | | | | | | |
| | | Nx7 peptide | | | | NTx | | | |
| | | Antibody | | | | | | | |
| | | S88230R | | 1H11 | | S88230R | | 1H11 | |
| | | Antibody concentration [μg/mL] | | | | | | | |
| | | 0.05 | 0.3 | 0.05 | 0.3 | 0.05 | 0.3 | 0.05 | 0.3 |
| Uric acid | 0% | 100% | 100% | 100% | 100% | 100% | 100% | 100% | 100% |
| concentration | 0.02% | 97% | 99% | 99% | 97% | 91% | 96% | 95% | 93% |
| | 0.05% | 100% | 98% | 100% | 95% | 87% | 92% | 84% | 83% |
| | 0.10% | 102% | 98% | 100% | 100% | 73% | 84% | 65% | 66% |

### Liquid-Phase Competitive ELISA

Pierce Streptavidin Coated Plates (manufactured by Thermo Scientific) were washed three times with PBST. Then, 50 μL of 0.1 μg/mL biotin-labeled Nx7 peptide was dispensed into each well and the resulting was allowed to stand at room temperature for 1 hour. After washing each well three times with PBST, 25 μL of a urine sample (BizCom Japan, Inc.) from an osteoporosis patient diluted 5 times with 20 mM HEPES, pH 7.6 containing 0, 0.03 or 0.08% uric acid, and 25 μL of the S88230R antibody or the 1H11 antibody with a concentration of 0. 1 μg/mL was sequentially dispensed, mixed, and allowed to stand at room temperature for 1 hour. After dispensing the secondary antibody, the same operations as for the Nx7 peptide solid-phase ELISA in Example 1 were performed. The absorbance ratios in measurements of the samples were calculated with the absorbance in measurement of a sample without uric acid being defined as 100%.

The results are shown in Table 2. In the measurement using the S88230R antibody, the absorbance change was small in response to an increase in the uric acid concentration, but in the measurement using the 1H11 antibody, the absorbance significantly increased in response to an increased in the uric acid concentration. It is presumed that the NTx measurement system using the S88230R antibody enables measurements without being affected by the uric acid concentration in the sample.

TABLE 2

| | | Absorbance ratio | | | |
|---|---|---|---|---|---|
| | | Antibody | | | |
| | | S88230R | | 1H11 | |
| | | Urine sample | | | |
| | | Sample 1 | Sample 2 | Sample 1 | Sample 2 |
| Uric acid | 0% | 100% | 100% | 100% | 100% |
| concentration | 0.03% | 104% | 99% | 108% | 105% |
| | 0.08% | 106% | 104% | 120% | 121% |

### Example 2 Antibody Specificity Test 1

### Peptide Competitive ELISA

After washing Pierce Streptavidin Coated Plates (manufactured by Thermo Scientific) three times with PBST, 50 μL of 0.1 μg/mL biotin-labeled Nx7 peptide was dispensed into each well and the resulting was allowed to stand at room temperature for 1 hour. After washing each well three times with PBST, 25 μL of the Nx2 or Nx7 peptide at a concentration of 0.03, 0.02, 0.08, 0.4, 2 or 10 mg/mL was dispensed into each well, followed by dispensing 25 μL of the S88230R antibody or the 1H11 antibody at a concentration of 0.15 μL/mL into each well, and the resulting was allowed to stand for 1 hour. After dispensing the secondary antibody, the same operations as for the Nx7 peptide-immobilized ELISA in Example 1 were performed.

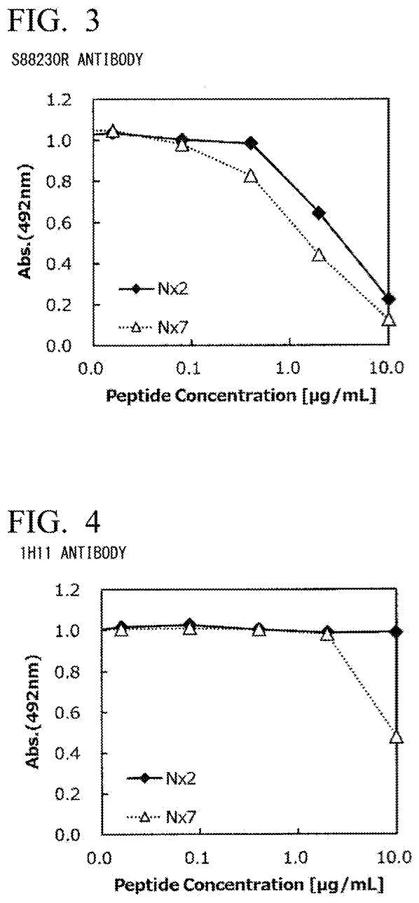

The results are shown in FIGS. 3 and 4. When an antibody reacts with a liquid phase peptide, the reaction between the liquid phase peptide and the antibody competes with the reaction between the solid phase peptide and the antibody, so that the absorbance decreases in response to an increase in the concentration of the liquid phase peptide. The S88230R antibody reacted with the immunogen Nx2, but the 1H11 antibody did not react with Nx2. Further, when Nx7 was added to the liquid phase, the absorbance for the S88230R antibody decreased more than that for the 1H11 antibody at a lower peptide concentration, suggesting that the S88230R antibody reacted more strongly with Nx7 than the 1H11 antibody. It was presumed that the difference in reactivity between the antibody and the peptide as described above caused the difference in the influence of uric acid on the measured value during NTx measurement.

### Example 3 Antibody Specificity Test 2

The reactivities of the S88230R antibody and 1H11 antibody with the peptides were evaluated in the same manner as in Example 2. The results are shown in Table 3. The S88230R antibody reacted with the Nx7-m3 peptide, in which the 4th amino acid G of the Nx7 peptide was replaced with S, and the Nx7-m5 peptide, in which the 7th amino acid V of the Nx7 peptide was replaced with L. In contrast, these peptides did not react with the 1H11 antibody. It was presumed that the S88230R antibody and the 1H11 antibody differ greatly in their reactivity in terms of the 4th amino acid G and the 7th amino acid V of the Nx7 peptide.

TABLE 3

| | | Reactivity between | |
|---|---|---|---|
| | | antibody and peptide | |
| Peptide | Amino acid sequence | S88230R | 1H11 |
| Nx2 | QYDGK (C) GVG | ++ | − |
| | (SEQ ID NO: 2) | | |
| Nx7 | JYDGKGVG | ++ | + |
| | (SEQ ID NO: 1) | | |
| Nx7-m1 | JFDGKGVG | − | − |
| | (SEQ ID NO: 4) | | |
| Nx7-m2 | JYEGKGVG | +/− | + |
| | (SEQ ID NO: 5) | | |
| Nx7-m3 | JYDSKGVG | + | − |
| | (SEQ ID NO: 6) | | |
| Nx7-m4 | JYDGKSVG | − | − |
| | (SEQ ID NO: 7) | | |
| Nx7-m5 | JYDGKGLG | +++ | − |
| | (SEQ ID NO: 8) | | |
| Nx7-m6 | JYDGKGVS | − | − |
| | (SEQ ID NO: 9) | | |

−: Not reacted, +/−: Weakly reacted, +: Reacted, ++:
Strongly reacted, +++: Very strongly reacted

### Example 4 Evaluation of Influence on Dilution Measurement

### ECLIA Measurement Using S88230R Antibody

Preparation of Nx7 Peptide-immobilized Magnetic Particles

After washing 100 μL of 30 mg/mL Streptavidin/solid-phase magnetic particles three times with PBS, followed by completely removing PBS, 600 μL of a biotin-labeled Nx7 peptide solution dissolved in PBS at a concentration of 3.3 g/mL was added, and the resulting was stirred at 25° C. for 2 to 3 hours. After washing the obtained magnetic particles three times with magnetic particle storage solution (50 mM HEPES, 1% BSA, 150 mM NaCl, 2 mM EDTA-4Na, 0.01% Tween 20, pH 7.2), the magnetic particles were suspended in 300 μL of magnetic particle storage solution to obtain a suspension of Nx7 peptide-immobilized magnetic particles. The Nx7 peptide-immobilized magnetic particle suspension was adjusted to a concentration of 0.05 mg/mL with R2 reagent (50 mM HEPES, 1% BSA, 150 mM NaCl, 2 mM EDTA-4Na, 0.01% Tween 20, pH 7.01% Tween 20, pH 7.2), and subjected to ECLIA measurement.

### Preparation of Ruthenium-Labeled S88230R Antibody

To 1 mL of 1 mg/mL S88230R antibody, 68 μL of 10 mg/mL Ruthenium (II) Tris(bipyridyl)-NHS Ester (dissolved in DMSO) was added, followed by stirring at room temperature for 30 minutes. Then, 50 μL of 2 mol/L glycine was added, and the resulting was stirred at room temperature for 10 minutes. From the obtained reaction solution, unreacted antibodies and ruthenium complexes were removed using Sephadex G-25 to obtain a ruthenium-labeled S88230R antibody.

### Preparation of Calibrator and Sample

The Nx7 peptide was dissolved in R1 reagent (50 mM HEPES, 1% BSA, 150 mM NaCl, 2 mM EDTA-4Na, 0.01% Tween 20, non-specific reaction inhibitor, pH 7.2) to a concentration of 250 ng/mL to obtain a standard product. Solutions in which the standard product was diluted 1, 2, 4, 8, 16, 32, 64, and 128 times with the R1 reagent were prepared and used as calibrators. As samples, either undiluted urine specimens or urine specimens diluted with predetermined solutions were used.

### ECLIA Measurement

Measurement of NTx by ECLIA was carried out using an ECLIA automated analyzer “Picolumi III”. 20 μL each of the calibrator and sample were injected into reaction tubes. 50 μL of ruthenium-labeled S88230R antibody adjusted to a concentration of 0.1 μg/mL with the RI reagent was injected into each reaction tube, and the resulting was stirred. 25 μL of 0.05 mg/mL Nx7 peptide-immobilized magnetic particles were injected into each reaction tube, and the resulting was allowed to react for 10.5 minutes. The liquid in the reaction tube was removed by suction, and the magnetic particles were washed with 350 μL of Picolumi BF washing solution (manufactured by Sekisui Medical Co., Ltd.). 300 μL of a luminescent electrolytic solution (manufactured by Sekisui Medical Co., Ltd.) was injected into the reaction tube, and the beads were guided to a flow cell electrode to measure the luminescence. From the measurement results of the calibrators, a calibration curve was created using the Logit-Log linear equation, and the measurement values for the samples were calculated. In this process, the measured value of the sample diluted with urine was calculated by subtracting the NTx value derived from the urine used for dilution from the actual measured value.

### Osteomark Measurement

NTx was measured using Osteomark kit for cross-linked N-telopeptides of type I collagen (Abbott Diagnostics Medical Co., Ltd.) according to the product's package insert. The standard product included in the kit was used, and the sample used was the same as that used for the ECLIA measurement. In this process, the measured value of the sample diluted with urine was calculated by subtracting the NTx value derived from the urine used for dilution from the actual measured value.

Using the ECLIA reagent and Osteomark of this example, 25 cases of osteoporosis patient-derived urine (BizCom Japan, Inc.) diluted 10 times with urine, physiological saline or uric acid diluent solution (0.15% uric acid, 20 mM HEPES, 150 mM NaCl, pH 7.6) were measured. The dilution recovery rates for dilution with the respective dilution solutions were calculated with the measured value for dilution with urine being defined as 100%. The results are shown in Table 4. The dilution measurement with physiological saline using the ECLIA reagent resulted in a value as high as 129% in terms of the average value of dilution recovery rate, but the dilution recovery rate values for the various samples were within ±20% of the average value, indicating that the variation in the measured values was very small. When the ECLIA reagent was used for dilution measurement with a uric acid diluent solution, both the recovery rate and the variation in recovery rate were good. On the other hand, when dilution measurements using physiological saline and uric acid diluent solution were performed with Osteomark which was prescribed in its package insert to use urine for diluting samples, the average values of dilution recovery rates were 101% and 118%, which were satisfactory, but the dilution recovery rates for the samples varied considerably. In the case of the ECLIA reagent of this example, the influence of the uric acid value of each sample on the measured value is negligible, which is presumed to be the reason for small variation of the measured values even when the uric acid value varied by the dilution measurements. Therefore, the ECLIA reagent of this example allows dilution measurement using a dilution solution other than urine by multiplying the measured value in the dilution measurement by a certain correction coefficient. This eliminates the need for cumbersome operation of post-measurement subtraction of the NTx value of the urine used for dilution, which is necessitated in dilution measurement with urine. As a result, it becomes possible to improve the convenience of NTx measurement. In addition, addition of uric acid to the diluent solution enables dilution 5 measurements without correction of measured values.

TABLE 4
Dilution recovery rate

| | ECLIA measurement | | Osteomark measurement | |
|---|---|---|---|---|
| | Saline | Uric acid | Saline | Uric acid |
| Sample | dilution | dilution | dilution | dilution |
| Sample 1 | 139% | 89% | 88% | 106% |
| Sample 2 | 140% | 91% | 88% | 82% |
| Sample 3 | 128% | 86% | 74% | 96% |
| Sample 4 | 132% | 87% | 99% | 99% |
| Sample 5 | 123% | 89% | 118% | 125% |
| Sample 6 | 120% | 80% | 102% | 100% |
| Sample 7 | 122% | 82% | 94% | 110% |
| Sample 8 | 131% | 91% | 89% | 98% |
| Sample 9 | 128% | 73% | 89% | 119% |
| Sample 10 | 121% | 80% | 91% | 121% |
| Sample 11 | 121% | 82% | 114% | 92% |
| Sample 12 | 152% | 95% | 133% | 171% |
| Sample 13 | 113% | 92% | 123% | 129% |
| Sample 14 | 133% | 95% | 119% | 119% |
| Sample 15 | 134% | 101% | 144% | 182% |
| Sample 16 | 121% | 97% | 105% | 133% |
| Sample 17 | 134% | 79% | 81% | 105% |
| Sample 18 | 117% | 92% | 96% | 114% |
| Sample 19 | 122% | 87% | 92% | 105% |
| Sample 20 | 125% | 91% | 105% | 116% |
| Sample 21 | 147% | 94% | 121% | 127% |
| Sample 22 | 145% | 90% | 88% | 123% |
| Sample 23 | 118% | 92% | 104% | 130% |
| Sample 24 | 131% | 97% | 85% | 111% |
| Sample 25 | 136% | 108% | 93% | 132% |
| Average value | 129% | 90% | 101% | 118% |
| Maximum value | 152% | 108% | 144% | 182% |
| Minimum value | 113% | 73% | 74% | 82% |
| Max − Min | 39% | 35% | 70% | 99% |

### Example 5 Antibody specificity test 3

Each of the peptides listed in Table 5 was dissolved in the RI reagent of Example 3 so as to prepare 70 μL of a solution containing the peptide at a concentration of 0, 35.6, 140 or 570 ng/mL and the ruthenium-labeled S88230R antibody at a concentration of 0.1 μg/mL, which was then measured with the ECLIA reagent. In addition, peptide solutions prepared at concentrations of 0, 0.1, 1.0 and 10 μg/mL in the same manner as in Example 2 were measured with Osteomark. Based on the measured values for the peptides, the reactivity between the antibodies and peptides included in the measurement systems was evaluated. The results are shown in Table 5. The reactivities between the S88230R antibody and the Nx7, Nx7-m3 and Nx7-m5 peptides were stronger than the reactivities between the antibody contained in Osteomark and these peptides. The Nx7-m3 peptide is a peptide in which the 4th amino acid G of the Nx7 peptide is replaced with S, and the Nx7-m5 peptide is a peptide in which the 7th amino acid V of the Nx7 peptide is replaced with L. These replacements had only a minor effect on the reactivity between the peptides and the S88230R antibody. Therefore, it is presumed that the effects of the present invention can be obtained even when a peptide in which the 4th and/or 7th amino acid(s) of the Nx7 peptide are/is substituted is used as a peptide for detection.

TABLE 5

| | | Reactivity between | |
|---|---|---|---|
| | | antibody and peptide | |
| | Amino acid | | Osteomark |
| Peptide | sequence | S8823OR | antibody |
| Nx7 | JYDGKGVG | +++ | ++ |
| Nx7-m1 | JFDGKGVG | +/− | − |
| Nx7-m2 | JYEGKGVG | + | ++ |
| Nx7-m3 | JYDSKGVG | ++ | − |
| Nx7-m4 | JYDGKSVG | + | + |
| Nx7-m5 | JYDGKGLG | +++ | + |
| Nx7-m6 | JYDGKGVS | +/− | − |

−: Not reacted, +/−: Weakly reacted, +: Reacted,
++: Strongly reacted, +++: Very strongly reacted

### Industrial Applicability

The present invention can provide a NTx measuring method and a NTx measuring kit, which are easier in operation and enable NTx measurement with higher accuracy.

1. An immunoassay method for cross-linked N-telopeptide of type I collagen, comprising:
a step of contacting a biological sample with an antibody or antibody fragment thereof that binds to a peptide fragment having an amino acid sequence represented by JYDGKGVG (SEQ ID NO: 1).

2. The immunoassay method according to claim 1, wherein the antibody or the antibody fragment thereof binds to a peptide fragment having an amino acid sequence represented by QYDGK(C)GVG (SEQ ID NO: 2).

3. The immunoassay method according to claim 1, wherein the antibody or the antibody fragment thereof is a monoclonal antibody or an antibody fragment thereof.

4. The immunoassay method according to claim 1, wherein the biological sample is urine, blood, plasma, or serum.

5. The immunoassay method according to claim 1, wherein a concentration of uric acid contained in a measurement system in the step of contacting is 0.001 to 0.1% by mass.

6. The immunoassay method according to claim 1, wherein the step of contacting is a step of bringing the biological sample into contact with the antibody or the antibody fragment thereof in the presence of a peptide fragment for detection which has an amino acid sequence represented by JYDX₁KGX₂G (SEQ ID NO: 3), wherein the X₁ and the X₂ are predetermined amino acids,
wherein the peptide fragment for detection is bound to a solid phase or a labeling substance, and
the antibody or the antibody fragment thereof is allowed to bind to the peptide fragment for detection.

7. The immunoassay method according to claim 6, wherein the X₁ is glycine or serine, and the X₂ is valine or leucine.

8. The immunoassay method according to claim 1, which further comprises a step of measuring a signal generated by the labeling substance,
wherein the peptide fragment for detection is bound to a solid phase, and
the antibody or the antibody fragment thereof is indirectly or directly bound to the labeling substance.

9. The immunoassay method according to claim 8, wherein the solid phase is magnetic particles and the labeling substance is a ruthenium complex.

10. An immunoassay kit for cross-linked N-telopeptide of type I collagen in a biological sample, comprising an antibody or an antibody fragment thereof that binds to a peptide fragment having an amino acid sequence represented by JYDGKGVG (SEQ ID NO: 1).

11. The immunoassay kit according to claim 10, wherein the antibody or the antibody fragment thereof binds to a peptide fragment having an amino acid sequence represented by QYDGK(C)GVG (SEQ ID NO: 2).

12. The immunoassay kit according to claim 10, wherein the antibody or the antibody fragment thereof is a monoclonal antibody or an antibody fragment thereof.

13. The immunoassay kit according to claim 10, wherein the biological sample is urine, blood, plasma, or serum.

14. The immunoassay kit according to claim 10, further comprising a peptide fragment for detection containing an amino acid sequence represented by
(SEQ ID NO: 3)
JYDX₁KGX₂G,
wherein the X₁ and the X₂ are predetermined amino acids, and
the antibody or the antibody fragment thereof binds to the peptide fragment for detection.

15. The immunoassay kit according to claim 14, wherein the X₁ is glycine or serine, and the X₂ is valine or leucine.

16. The immunoassay kit according to claim 10, wherein the peptide fragment for detection is bound to a solid phase, and the antibody or the antibody fragment thereof is indirectly or directly bound to the labeling substance.

17. The immunoassay kit according to claim 16, wherein the solid phase is magnetic particles and the labeling substance is a ruthenium complex.

18. An antibody or antibody fragment thereof that binds to a peptide fragment having an amino acid sequence represented by JYDGKGVG (SEQ ID NO: 1).

19. The antibody or antibody fragment thereof according to claim 18, which binds to a peptide fragment having an amino acid sequence represented by QYDGK(C)GVG (SEQ ID NO: 2).

20. The antibody or antibody fragment thereof according to claim 18, which is a monoclonal antibody.

21. A solid phase for use in the immunoassay method of claim 1, which has a peptide fragment for detection bonded thereto, wherein the peptide fragment contains an amino acid sequence represented by JYDX₁KGX₂G (SEQ ID NO: 3) or a partially modified sequence thereof, wherein the X₁ and the X₂ are predetermined amino acids.

22. The solid phase according to claim 21, which is magnetic particles.
